(19) United States
(12) Patent Application Publication
(10) Pub. No.: US 2012/0289822 A1
(43) Pub. Date: Nov. 15, 2012

(54) APPARATUS AND METHOD FOR LORENTZ-ACTIVE SHEATH DISPLAY AND CONTROL OF SURGICAL TOOLS

(75) Inventors: Yehoshua Shachar, Santa Monica, CA (US); Bruce Marx, Ojai, CA (US); Leslie Farkas, Ojai, CA (US); David Johnson, West Hollywood, CA (US); Laszlo Farkas, Ojai, CA (US)

(73) Assignee: MAGNETECS, INC., Inglewood, CA (US)

(21) Appl. No.: 13/450,148

(22) Filed: Apr. 18, 2012

Related U.S. Application Data
(63) Continuation of application No. 12/099,079, filed on Apr. 7, 2008, now abandoned.

(51) Int. Cl. A61B 1/32; A61B 6/00

(52) U.S. Cl. 600/424; 600/202

(57) ABSTRACT

The Lorentz-Active Sheath (LAS) serves as a conduit for other medical devices such as catheters, balloons, biopsy needles, etc. The sheath is inserted through a vein or other body orifice and is guided into the area of the patient where the operation is to be performed. The position and orientation of the LAS is tracked via an industry standard position detection system which senses electrical signals that are emitted from several electrodes coupled to the LAS. The signals received from the LAS are used to calculate an accurate and reliable assessment of the actual position of the LAS within the patient. The electrode signals also serve to create a reference frame which is then used to act as a motion compensation filter and fiducial alignment system for the movement of the LAS-hosted medical tool.

### CROSS-REFERENCE TO RELATED APPLICATIONS

This application is a continuation of U.S. patent application Ser. No. 12/099,079, filed Apr. 7, 2008, the entirety of which is incorporated herein by reference and is to be considered part of this specification.

### BACKGROUND OF THE INVENTION

1. Field of the Invention

The invention relates to the field of determining the location, orientation, and movement of an invasive medical device within a patient while compensating for undesired tool and patient motion.

2. Description of the Related Art

Medical sheaths have long been used to introduce a variety of medical tools into a patient during an operation. Typically, the sheath is inserted into the patient via a vein or other orifice and is manipulated until it has reached its target location such as a ventricle of the heart. Invasive medical tools such as catheters, balloons, and biopsy needles are then deployed through the sheath in order to work on the patient.

While the prior art has been successful in treating many patients, the techniques of the prior art are not without their drawbacks and difficulties. Fluoroscopy or x-rays can be used to image fiducial points such the radio-opaque markers or rings that have been placed on the medical sheath and transmits them to a display. The physician is then able to view and analyze the sheath's current location and orientation of the sheath's distal tip. If the sheath is in the wrong area, needs to be adjusted, or has been dislocated, the sheath must be moved or recovered and then another medical image must be taken. This process is repeated until the sheath has reached the desired location.

The prior art does not provide a consistent, and reliable fix on the location of the sheath as well as maintaining a known orientation of the distal tip of the sheath. The prior art uses fiducial markers such as the ones presented above which can only be seen when using an ionizing field source such as an x-ray or CT scan and are useless when employed in a radar based navigation system.

Also not contemplated by the prior art is the use of a medical sheath as a motion compensation filter for the deployment of medical tools. The prior art has so far failed to employ the sheath itself for motion compensation.

### SUMMARY OF THE INVENTION

The system and methods described herein solve these and other problems by adding navigation electrodes to the medical sheaths that are used to deploy catheters, balloons, biopsy needles, and other medical tools within a patient during invasive surgery. The system is capable of continuously determining the location and position of the sheath's distal tip in up to six degrees of freedom or even more and within 1 mm of the tip's actual position within a patient. Navigational electrodes that emit an electrical signal to a nearby receiver are located at or near the tip of the sheath and along down the shaft to determine key locations of the sheath and to serve as a global fiducial reference frame. This reference frame that is created by the sheath is then used to compensate for changes in the patient's or local organ's orientation.

One embodiment includes a system that can continually determine the position and location of the distal tip of a medical sheath and track its movements in six degrees of freedom as it is manipulated through a patient while compensating for the movement of the patient or organ it is working in without the use of fluoroscopy or other medical imaging devices that use an ionizing field source.

In one embodiment, a Lorentz-Active Sheath (LAS) is used during invasive surgery in a moving organ such as the heart where medical tools such as catheters, biopsy needles, balloons, and the like are required. The position of the LAS electrodes are tracked in the presence of dynamic variables such as the mechanical contraction and repolarization of the heart muscle. The data acquired from the tracking of the LAS electrodes is then used to produce a reference frame of the sheath as it moves in conjunction with the patient or moving tissue.

In one embodiment, the average position and orientation of the LAS distal tip is continuously determined with respect to the previously measured positions of the tip over a specific time period. This process provides a reference position and orientation that is later used for compensating the motion of the LAS-hosted medical tools.

In one embodiment, the motion of the LAS is defined with respect to the aforementioned average position. This process provides a position and orientation error value that is later incorporated into motion compensation and fiducial alignment modalities.

In one embodiment, the position and orientation error values of the LAS are used to subtract the motion of the LAS from the motion of the LAS-hosted medical tool. This in effect along with the previous three embodiments forms a motion compensation filter and provides a stable fiducial reference for tool position control systems and thus provides the operating physician with an accurate assessment of the sheath's true position within the patient.

In one embodiment, the positions of the LAS navigation electrodes are used to determine a six-degree of freedom reference frame.

In one embodiment, the reference frame (e.g., six degrees) that was created from the LAS navigation electrodes in the previous embodiment is used to track changes in the patient's or local organ's orientation.

While the apparatus and method is described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings.

### BRIEF DESCRIPTION OF THE DRAWINGS

FIG. 1 is an isometric diagram of the Lorentz-Active Sheath (LAS) assembly.

FIG. 2 is a block diagram of the signals and systems that determine the Lorentz-Active Sheath position, position error, position compensation, and patient fiducial alignment.

FIG. 3 is a schematic diagram of the motion compensation vectors.

FIG. 4 is a schematic diagram of the LAS electrodes used to determine the fiducial quaternions and position reference.

FIG. 5 is a schematic diagram of the patient fiducial alignment quaternions.

FIG. 6 is a block diagram of an embodiment of the invention which incorporates the Lorentz-Active Sheath into a Catheter Guidance Control and Imaging (CGCI) system and depicts its function of providing a reference between the catheter, the patient, the fiducial alignment system, and a console catheter data filtering system.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION

In general, the Lorentz-Active Sheath (LAS) serves as a conduit for other medical devices such as catheters, balloons, biopsy needles, etc. The sheath is inserted through a vein or other body orifice and is guided into the area of the patient where the operation is to be performed. The position and orientation of the LAS is tracked via a conventional position detection system which senses electrical signals that are emitted from several electrodes coupled to the LAS. The signals received from the LAS are used to calculate an accurate and reliable assessment of the actual position of the LAS within the patient. The electrode signals also serve to create a reference frame which is then used to act as a motion compensation filter and fiducial alignment system for the movement of the LAS-hosted medical tool.

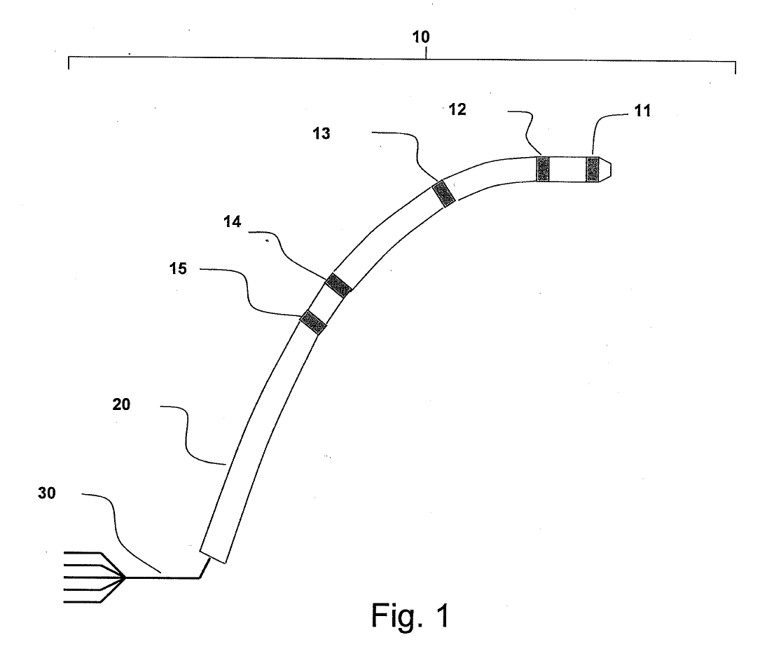

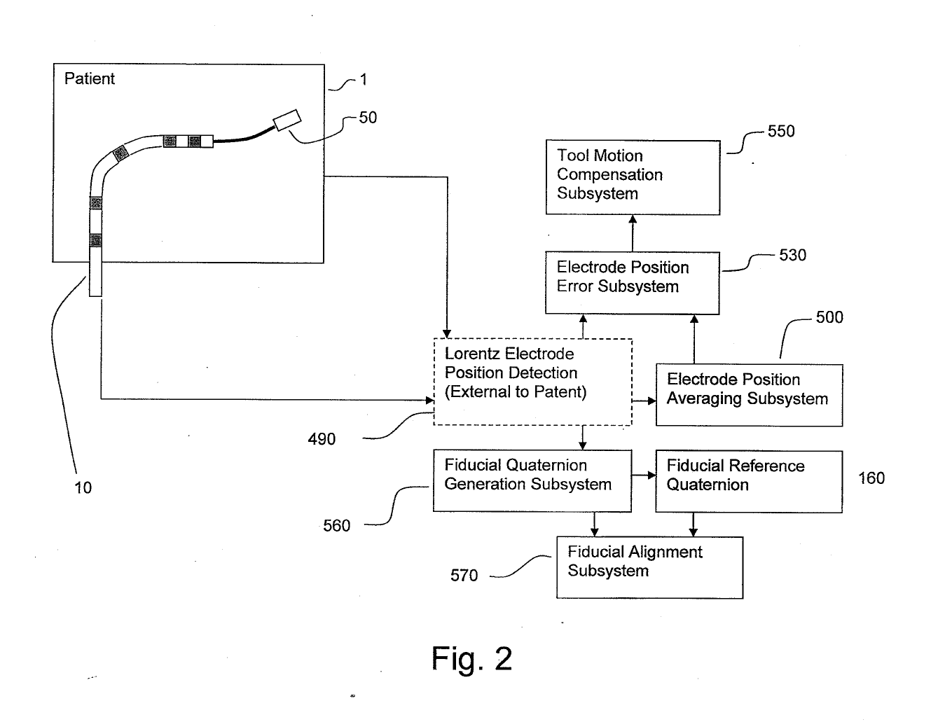

FIG. 1 is an isometric diagram of the LAS assembly 10. Detection system-sensitive electrodes 11-15 are integrated into the LAS shaft 20. The electrodes 11-15 are used to generate electrical signals which are sensed by a position detection system 490 shown in FIG. 2. The electrodes 11-15 can sensors, such as, for example, impedance sensors, radar sensors, hall-effect sensors, etc. and/or sources, such as, for example, radio-frequency sources, radio-frequency coils, piezoelectric rings, etc.

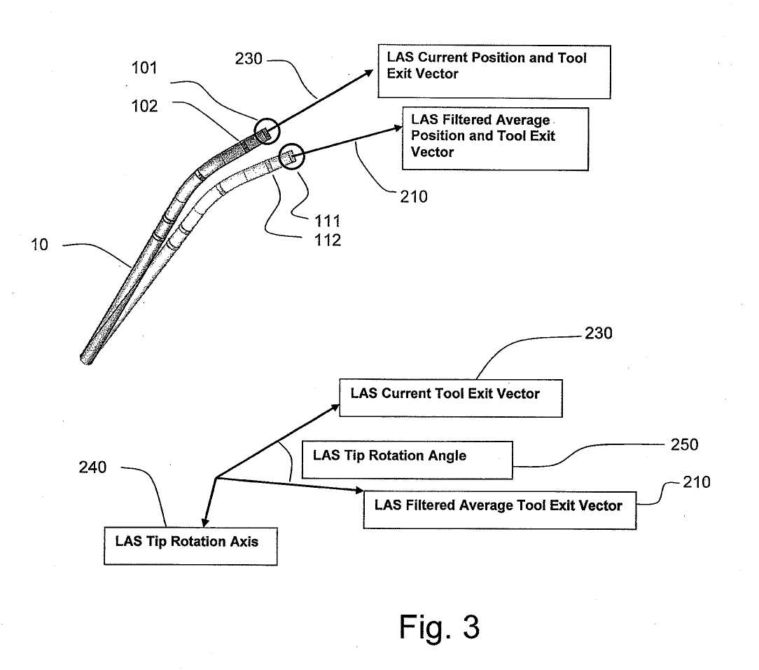

The two most distal electrodes 11 and 12 on shaft 20 are used to determine the tool exit position and tool exit direction 230 from the LAS 10 as illustrated in FIG. 3. FIG. 1 also shows that electrodes 11-15 are connected to the position detection system 490 by embedded electrode wires 30 which are attached to a coupling connector (not shown).

In one embodiment, one or more electrodes 11-15 sense the electrical signals transmitted between a plurality of surface electrode patches. The system collects electrical data from the one or more electrodes 11-15 uses this information to track or navigate their movement and construct three-dimensional (3-D) models of the tissues.

In one embodiment, one or more electrodes 11-15 sense the electrical signals transmitted between three pairs of EnSite NavX surface electrode patches, such as, for example the EnSite NavX surface electrode patches used in connection with the EnSite System. The system collects electrical data from the one or more electrodes 11-15 and uses this information to track or navigate movement of the one or more electrodes 11-15 and construct three-dimensional (3-D) models of the chamber.

FIG. 2 is a block diagram of the signals and systems used to determine the position, position error, position compensation, and patient fiducial alignment of the LAS 10.

The LAS 10 is inserted into a patient 1 through a medical incision or body orifice. A LAS-hosted medical tool 50 such as a catheter, balloon, biopsy needle, or any other medical device that may be required during an invasive operation is inserted through the LAS 10 and deployed into the patient volume in which the operation is to occur. The detection system-sensitive electrodes 11-15 that are provided to the LAS 10, the LAS-hosted tool 50, and patient 1 are provided to the position detection system 490 by standard connectors and patches (not shown).

In one embodiment, the LAS 10 is used to act as a motion compensation device and subtract unwanted motion of the sheath from the motion of the currently deployed LAS-hosted medical tool 50. The position detection system 490 provides the current positions of the electrodes located on the LAS 10 as well as the positions of the electrodes located on the LAS-hosted medical tool 50 through a system of network communications and standard computer software interfaces. The position data of the LAS 10 that has been collected by the position detection system 490 is then sent to the Electrode Position Averaging Subsystem 500 as depicted in FIG. 2. The Electrode Position Averaging Subsystem 500 averages the positions of the electrodes located on LAS 10 over a select time period in order to obtain a stable baseline reference of the position of the LAS. This new averaged electrode position is then subtracted from the current electrode position provided by the position detection system 490 by the Electrode Position Error Subsystem 530. The error measurement that has been created by the Electrode Position Error Subsystem 530 is then sent to the Tool Motion Compensation Subsystem 550 which is employed to subtract unwanted sheath motion from the motion of the currently deployed LAS-hosted tool 50.

Further understanding of the process described above can be obtained by turning to the following example. As depicted in FIG. 1, detection system-sensitive electrodes 11 and 12 are located on the shaft 20 of the LAS 10. The position detection system 490 locates electrodes 11 and 12 and thus the sheath while the LAS is in the operating volume of the patient 1. The raw data that the position detection system 490 collects produces an image of the position of electrodes 11 and 12 and displays them as current LAS electrode positions 101 and 102 respectively as shown in FIG. 3. This process is then repeated a number of times over a specified time period.

After the position detection system 490 has found multiple data points for current electrode positions 101 and 102, the data for each electrode is then sent to the Electrode Position Averaging Subsystem 500. The Electrode Position Averaging Subsystem 500 begins to average the last n number of current positions obtained for each electrode using equation (1). For example, current electrode position 101 that was obtained originally from electrode 11 is averaged in the following manner:

LAS Average Electrode 101 Position=SUM(LAS Current Electrode 101 Positions)/n (1)

where n is the number of measurements taken. Once current electrode positions 101 and 102 have been applied to equation (1) and filtered, the average electrode positions 111 and 112 are obtained respectively as shown in FIG. 3. It is to be expressly understood that any multitude or plurality of current electrode positions may be analyzed in this manner.

Current electrode positions 101 and 102 and filtered average electrode positions 111 and 112 can then be used to calculate the position and exit orientation of a medical tool that is to be deployed by the LAS 10. The exit orientation or exit vector of a deployed medical tool is found by normalizing the difference in position between the two most distal electrodes 101 and 102 using equation (2):

LAS Tool Exit Vector=(LAS Electrode 101 Position−LAS Electrode 102 Position)/|LAS Electrode 101 Position−LAS Electrode 102 Position| (2)

This equation thus produces a current exit vector 230 for a deployed medical device as shown in FIG. 3. Equation (2) is also applied to the filtered average electrode positions 111 and 112 to produce an average exit vector 210 for a deployed medical device also shown in FIG. 3. This newly obtained exit vector gives the operating physician a clear and reliable reading on exactly where his instruments are within the patient volume and in what orientation the instruments are traveling in. As shown from the description above, if the LAS is in the wrong position or is being manipulated into the wrong direction, the physician may quickly and easily re-position the LAS in real time without the use of fluoroscopy or other medical images that use an ionizing field source.

After the position data has passed from the Electrode Position Averaging Subsystem 500 and through the Electrode Position Error Subsystem 530, it is relayed to the Tool Motion Compensation Subsystem 550 where the motion of the sheath is subtracted from the motion of the LAS-hosted tool 50 to produce an extremely accurate and consistent assessment of the medical tool's location within the patient volume in six degrees of freedom. In order to accomplish this, the motion compensation due to the displacement of the LAS tip is performed by subtracting the LAS motion with respect to the average tip position of the LAS which is given by equation (3):

Tool Position′=Tool Position−[LAS Electrode 101 Position−LAS Filtered Average Electrode 111 Position] (3)

Similarly, motion compensation due to tip rotation of the LAS is performed by un-rotating the tool-to-LAS tip position vector using equation (4).

Tool Position Vector=Tool Position−LAS Electrode 101 Position (4)

The LAS filtered average tool exit vector 210 is crossed with the LAS current tool exit vector 230 to give the LAS tip rotation axis 240 given in equation (5) and as shown in FIG. 3.

LAS Tip Rotation Axis=(LAS Filtered Average Tool Exit Vector)×(LAS Current Tool Exit Vector) (5)

The dot product of the same two vectors in equation (6) gives the LAS tip rotation angle 250.

LAS Tip Rotation Angle=(LAS Filtered Average Tool Exit Vector)·(LAS Current Tool Exit Vector) (6)

The tool position vector is then rotated about the LAS tip rotation axis 240, the result of equation (5), by the negative of the LAS tip rotation angle 250, the result of equation (6), to give the adjusted tool position vector using standard rotation matrices and equation (7).

Tool Position′(angle)=Tool Position rotated about (LAS Tip Rotation Axis) by−(LAS Tip Rotation Angle) (7)

Finally, the total compensation due to the position and angle shifts of the LAS may be found by combining equations (7) and (3) into equation (8).

Tool Position′(total)=Tool Position′(angle)−[LAS Electrode 101 Position−LAS Filtered Average Electrode 111 Position] (8)

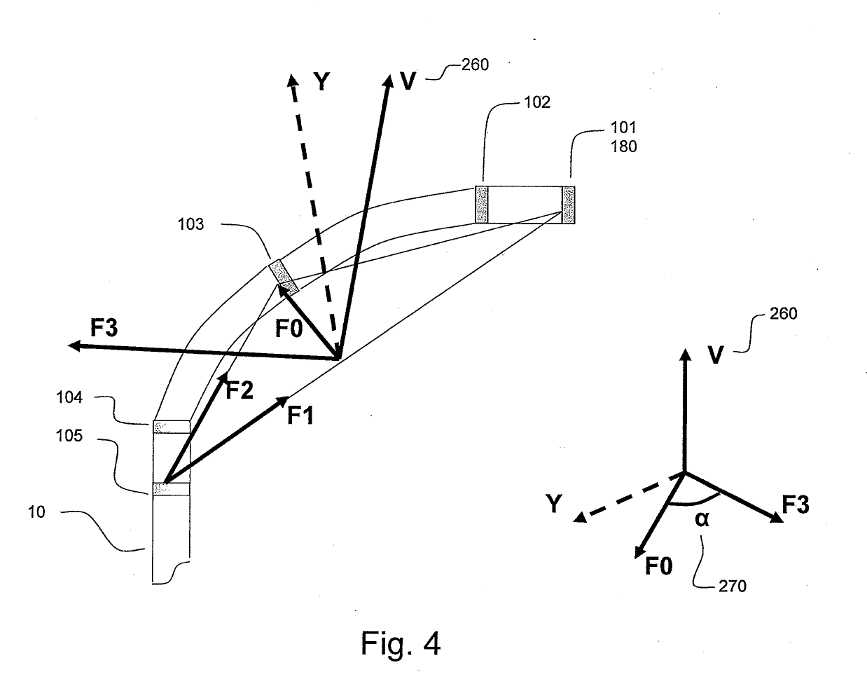

In one embodiment, the LAS device is used to track local tissue motion and alignment. In FIG. 2, the current electrode positions 101 and 102 and any other electrodes that may be placed on the shaft 20 of the LAS that are generated by the Position detection system 490 are sent to the LAS Fiducial Quaternion Generation Subsystem 560 which in turn generates a six-degree of freedom reference set of the LAS Current Fiducial Reference Quaternion 160 and LAS Current Fiducial Position 180 (shown in FIG. 4). These two newly acquired data sets are then used by the LAS Fiducial Alignment Subsystem 570 to track the motion and alignment of local tissue.

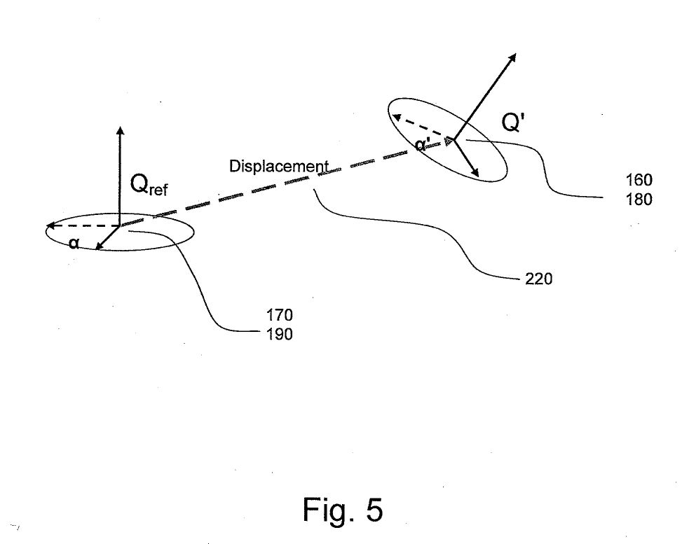

FIG. 4 is a schematic diagram of the LAS electrodes used to derive the fiducial quaternion and position reference. The position of the first current electrode 101 defines the LAS Current Fiducial Position 180. Current electrode 101 along with current electrodes 103 and 105 form a fiducial reference triangle. The LAS Current Fiducial Quaternion 160 (shown in FIG. 5) is determined by the vector normal to the triangle plane and the rotation of the triangle with respect to the patient axis Y, projected into the fiducial plane.

The fiducial triangle orientation FO, is calculated by using basic trigonometry in equation (9).

F0=(LAS Electrode 103 Position)−(LAS Electrode 101 Position)/2−(LAS Electrode 105 Position)/2 (9)

Two additional fiducial reference vectors, F1 and F2, are needed to determine the fiducial triangle and are derived from equations (10) and (11).

F1=(LAS Electrode 101 Position)−(LAS Electrode 105 Position) (10)

F2=(LAS Electrode 103 Position)−(LAS Electrode 105 Position) (11)

The cross product of vectors F1 and F2, shown in equation (12), is then normalized by equation (13) to give the LAS Fiducial Vector 260 as shown in FIG. 4.

vs=(F1×2)/|F1| |F2| (12)

v=vs/|vs| (13)

The LAS Fiducial Vector 260 is then crossed in equation (14) with the patient axis Y to give a reference vector in the fiducial triangle plane, F3, which is then used in equation (15) to calculate the LAS Fiducial Rotation Angle α 270.

F3=(v×(Y−Axis)) (14)

α=arc cosine(F3·F0/|F0|) (15)

The LAS Current Fiducial Quaternion 160 of FIG. 5 is then calculated by the standard method to give the four-element quaternion vector shown in equation (16).

Q={v cos(α/2), sin(α/2)}=<vx cos(α/2), vy cos(α/2), vz cos(α/2), sin(α/2)> (16)

FIG. 5 is a schematic diagram of the patient fiducial alignment quaternions and fiducial reference displacement used to normalize patient motion to the reference position and orientation. The LAS Reference Fiducial Quaternion 170 is set to the LAS Current Fiducial Quaternion 160 when the patient is at the reference position. Also when the patient is at the reference position, the LAS Current Fiducial Position 180 becomes the LAS Reference Fiducial Position 190. Any deviation from this reference position and orientation may be used to normalize the system vectors between the new patient position and orientation, and the reference position and orientation.

Given the LAS Reference Fiducial Quaternion 170, LAS Reference Fiducial Position 190, LAS Current Fiducial Quaternion 160, and the LAS Current Fiducial Position 180, any vector V may be referenced back to the reference orientation by the standard quaternion algebra.

Vector V is defined in three dimensions with respect to the fourth by appending zero to the vector in equation (17). This is done whenever multiplying a vector by a quaternion using quaternion algebra.

V≡<x,y,z,0> (17)

Referencing V in current space to the reference orientation requires that it is rotated in the opposite direction by the current quternion and rotated by the reference. The standard rotation equation for the rotation of a vector by a quaternion is given in equation (18),

v′=qvq* (18)

where q* is the conjugate of the unit quaternion <−x, −y, −z, w>. To un-rotate the vector by Q then re-rotate the vector by Qr, the standard form is given in equation (19) below.

Vref=QrQ*vQQr* (19)

Referencing a vector V in reference space to the current orientation is done similarly in equation (20).

V=QQr*Vref QrQ* (20)

Converting a position in current space to reference space is done by rotating the relative position vector and then accounting for the displacement of the LAS Fiducial Position 220. The relative position vector, Prel, is calculated with respect to the LAS Current Fiducial Position 180 in equation (21) below.

Prel=P−(LAS Current Fiducial Position) (21)

Prel is then rotated into reference space by equation (22).

Prel′=QrQ*Prel QQr* (22)

P′ is then calculated in equation (23) by adding the reference position and subtracting the fiducial position change from the result obtained by equation (22).

P′=Prel′+LAS Fiducial Reference Pos−(LAS Current Fiducial Position−LAS Fiducial Reference Position) (23)

P′ will reflect the same relative position on the un-rotated patient as P in the current patient orientation.

To reference a position in reference space to current space, the same method is applied. The relative position vector Prel is calculated with respect to the LAS Reference Fiducial Position 190 in equation (24) below.

Prel=P−(LAS Reference Fiducial Position) (24)

Prel is then rotated into current space by equation (25).

Prel′=QQr*Prel QrQ* (25)

P′ is then calculated much like before by adding the reference position and subtracting the fiducial position change to the resultant of equation (25) as shown in equation (26).

P′=Prel′+LAS Current Fiducial Position−(LAS Reference Fiducial Position−LAS Current Fiducial Position) (26)

P′ will reflect the same relative position on the rotated patient as P in the reference patient orientation.

Once all the equations above have been solved by the Fiducial Alignment Subsystem 570, the operating physician can then track the movement of the LAS device in its relation to the surrounding patient operation volume. This feature of the device is extremely useful in circumstances where the LAS must be employed in an invasive surgery within a beating heart or other similar moving tissue. The fiducial alignment system allows the motion of the moving tissue to be tracked and anticipated and therefore, movement of the patient or the surrounding operation volume does not interfere or complicate the physician's procedure.

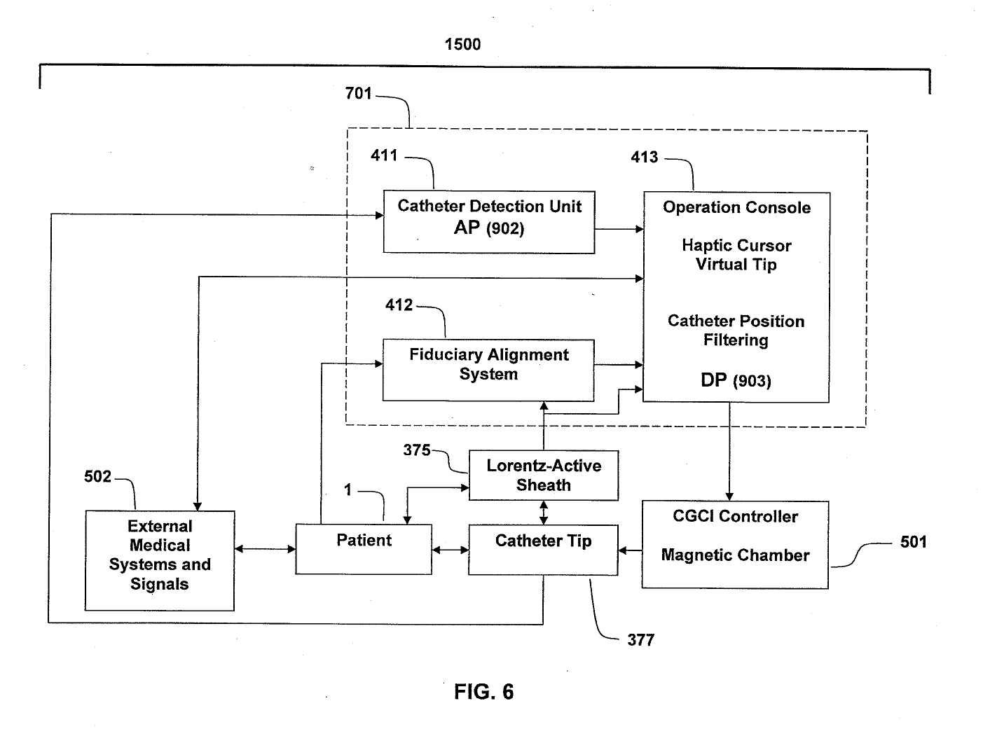

FIG. 6 is a block diagram of a CGCI unit 1500 that incorporates the Lorentz-Active Sheath into a Catheter Guidance Control and Imaging (CGCI) system. This combination provides a LAS reference coordinate set to the CGCI fiduciary alignment system 412 and data filtration routines of the CGCI operation console 413 in order to stabilize the undesired motion of the catheter tip 377 and align it within the patient 1.

The CGCI unit 1500 which includes a magnetic chamber along with an adaptive regulator, a joystick haptic device for operator control, and a method for detecting a magnetically-tipped catheter is described, for example in U.S. patent application Ser. No. 16/697,690 titled “Method and Apparatus for Controlling Catheter Positioning and Orientation” and is hereby incorporated by reference. A detailed description of the preferred embodiments using the Lorentz Active Sheath (LAS 375) in combination with the magnetic chamber forming the CGCI 1500 is noted by U.S. patent application Ser. No. 10/621,196 “Apparatus for Catheter, Guidance, Control, and Imaging”, U.S. patent application Ser. No. 11/331,781, “System and Method for Controlling Movement of a Surgical Too”, U.S. application Ser. No. 11/331,994, “Apparatus and Method for Generating a Magnetic Field”, U.S. application Ser. No. 11/331,485, “System and Method for Magnetic Catheter tip,” “System and Method for Radar Assisted Catheter Guidance and Control” U.S. application Ser. No. 10/690,472, titled, “System and Method for Radar Assisted Catheter Guidance and Control,”, U.S. application Ser. No. 11/140,475, “Apparatus and Method for Shaped Magnetic Field Control for Catheter, Guidance, Control and Imaging.”, U.S. application Ser. No. 11/362,542, “Apparatus for Magnetically Deployable Catheter with Mosfet Sensors and Method for Mapping and Ablation.”, hereby incorporated by reference. The above magnetic navigation system 1500 is further augmented by the Lorentz Active Sheath 375 so as to render the error generated by the dynamic movements of the mural to be filtered using the sensory ring 11, 12, 13, 14, and 15 and the computer software algorithm forming a filtering technique such as, for example, a Kalman Filter.

In the present embodiment, the catheter tip 377 and Lorentz-Active Sheath 375 are being operated within the patient 1. The CGCI imaging and synchronization unit 701 detects the actual position (AP) 902 of the catheter tip 377 and the position and orientation of the LAS 375. The CGCI imaging and synchronization unit 701 filters and aligns the data and specifies a desired position (DP) 903 for the catheter tip 377 under operator input through the CGCI virtual tip 905. The CGCI catheter detection unit 411 remotely senses the actual position and orientation 902 of the catheter tip 377 and the LAS 375 with respect to the CGCI global coordinate system 100. The LAS provides the CGCI fiduciary alignment system 412 with an LAS current fiducial quaternion 160 and an LAS reference fiducial quaternion 170 to normalize the AP 902 under patient rotation and translation within the CGCI global coordinate system 100. The position and orientation of the LAS current fiducial quaternion 160 establishes the patient tissue reference position and orientation within the global coordinate system 100. The position and orientation of the LAS reference fiducial quaternion 170 is initialized at a known tissue position and orientation to normalize LAS 375 and catheter tip 377 coordinates to medical data and models, such as those provided by the external medical systems and signals 502. The desired position 903 is then specified in reference to such medical data and models as to allow the CGCI controller 501 to regulate between the actual position 902 and the desired position 903 within the local patient coordinate frame 200.

The LAS filtered average positions 110 are used by the CGCI operation console 413 to remove any undesired catheter tip motion due to the motion of the distal end of the LAS.

Under dynamic variables, such as mechanical contractions and repolarization of the heart muscle, the CGCI filtering of the catheter tip motion becomes a dominant concern. The CGCI fiducial alignment system 412 acts to filter the dynamic motion of the LAS current fiducial quaternions by limiting the fiducial alignments system's response to gross patient motion while at the same time not interfering with the use of the LAS as a QRS regiments filter for the actual position 902 of the catheter tip.

In the absence of dynamic variables, such as surgery in the brain, the CGCI fiducial alignment system 412 will dominate the normalization of the incoming AP values so as to maintain a precise alignment between the sensed positions, tissue, and acquired data models.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

For example, one skilled in the art may choose to imbed a large plurality of detection system-sensitive electrodes, such as ten or more, along the shaft of the LAS 10 to provide an even more accurate and precise motion compensation filter and fiducial alignment system. Additionally, one skilled in the art may also choose to use alternate devices other than electrodes to signal the position of the LAS device or use alternate means of receiving the signals other than a position detection system.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

What is claimed is:

1. A medical apparatus to be used during an invasive surgery comprising:
a sheath capable of deploying a multitude of medical tools and adapted for insertion into the body of a patient;
at least one electrode coupled to the sheath;
a position detection system coupled to the sheath capable of sensing said electrode coupled to the sheath;
a computer software program coupled to the position detection system capable of compensating for the unwanted motion of the sheath by subtracting said sheath motion from the motion of the sheath-hosted tool; and
a computer software program coupled to the position detection system capable of tracking the sheath's progress through the surrounding tissue of a patient by means of fiducial alignment.

2. The apparatus of claim 1 further comprising a plurality of electrodes coupled to the sheath.

3. The apparatus of claim 1 wherein the computer software program capable of compensating for unwanted sheath motion further comprises a subsystem coupled to the position detection system for averaging the electrode position.

4. The apparatus of claim 3 wherein the computer software program capable of compensating for unwanted sheath motion further comprises a subsystem for calculating the electrode position error coupled to both the position detection system and the subsystem for averaging the electrode position.

5. The apparatus of claim 4 wherein the computer software program capable of compensating for unwanted sheath motion further comprises a subsystem that compensates for tool motion coupled to the subsystem for calculating the electrode position error.

6. The apparatus of claim 1 wherein the computer software program capable of tracking the sheath's progress through the surrounding tissue of a patient by means of fiducial alignment further comprises a subsystem coupled to the position detection system that generates a fiducial quaternion.

7. The apparatus of claim 6 wherein the computer software program capable of tracking the sheath's progress through the surrounding tissue of a patient by means of fiducial alignment further comprises a fiducial alignment subsystem coupled to the subsystem that generates a fiducial quaternion.

8. The apparatus of claim 1 further comprising a means for incorporating the device into a catheter guidance control and imaging system.

9. A method of tracking and compensating for medical tool motion during an invasive surgery within the body a patient comprising:
inserting a medical sheath capable of deploying a multitude of medical tools comprising at least one electrode coupled to the sheath into an incision or other body orifice of the patient;
detecting the position and orientation of the electrode using a position detection system;
sending the data collected by the position detection system through a series of computer software subsystems that produce from a series of calculations a motion compensation filter for the sheath-hosted tool; and
sending the data collected by the position detection system through a series of computer software subsystems that track the motion of the sheath and sheath-hosted tool by a means of fiducial alignment.

10. The method of claim 9 wherein detecting the position and orientation of the electrode using a position detection system further comprises incorporating the device into a catheter guidance control and imaging system.

11. The method of claim 9 further comprising inserting a medical sheath capable of deploying a multitude of medical tools comprising a plurality of electrodes coupled to the sheath.

12. The method of claim 9 wherein sending the data collected by the position detection system through a series of computer software subsystems that produce from a series of calculations a motion compensation filter for the sheath-hosted tool further comprises providing a reference position and orientation for motion compensation for sheath-hosted tools by an average position and orientation of the sheath's distal end being continuously determined with respect to several measured positions over a set time period.

13. The method of claim 9 wherein sending the data collected by the position detection system through a series of computer software subsystems that produce from a series of calculations a motion compensation filter for the sheath-hosted tool further comprises providing a position and orientation error value by defining the sheath's motion with respect to an average position and orientation of the sheath.

14. The method of claim 9 wherein sending the data collected by the position detection system through a series of computer software subsystems that produce from a series of calculations a motion compensation filter for the sheath-hosted tool further comprises providing a stable fiducial reference for a tool position control system of the position detection system by subtracting position error values from the motion of the sheath-hosted tool.

15. The method of claim 9 wherein sending the data collected by the position detection system through a series of computer software subsystems that track the motion of the sheath and sheath-hosted tool by a means of fiducial alignment further comprises employing the electrode to determine a six-degree of freedom reference frame.

16. The method of claim 9 wherein sending the data collected by the position detection system through a series of computer software subsystems that track the motion of the sheath and sheath-hosted tool by a means of fiducial alignment further comprises employing a six-degree of freedom reference frame to track changes in a patient's or local organ's orientation.

17. The apparatus of claim 3 wherein the subsystem for averaging the electrode position further comprises a means for determining the average position of the distal electrode coupled to the sheath.

18. The apparatus of claim 3 wherein the subsystem for averaging the electrode position further comprises a means for determining the average exit vector of the deployed medical tool as it leaves the distal end of the sheath.

19. The method of claim 9 wherein sending the data collected by the position detection system through a series of computer software subsystems that track the motion of the sheath and sheath-hosted tool by a means of fiducial alignment further comprises a means for determining the average position of the distal electrode coupled to the sheath.

20. The method of claim 9 wherein sending the data collected by the position detection system through a series of computer software subsystems that track the motion of the sheath and sheath-hosted tool by a means of fiducial alignment further comprises a means for determining the average exit vector of the deployed medical tool as it leaves the distal end of the sheath.

21. A medical apparatus to be used during an invasive surgery comprising:
a sheath capable of deploying a multitude of medical tools and adapted for insertion into the body of a patient;
at least one electrode coupled to the sheath;
means for compensating for the unwanted motion of the sheath from the desired motion of the sheath-hosted tool; and
means for tracking the sheath and sheath-hosted tool through the surrounding tissue of a patient by means of fiducial alignment.

22. The apparatus of claim 21 wherein the sheath capable of deploying a multitude of medical tools and adapted for insertion into the body of a patient further comprises a plurality of electrodes coupled to the sheath.
